(19) United States
(12) Patent Application Publication
(10) Pub. No.: US 2025/0003939 A1
(43) Pub. Date: Jan. 2, 2025

(54) ANOMALY DETERMINATION APPARATUS, ANOMALY DETECTION APPARATUS, ANOMALY DETERMINATION METHOD, AND STORAGE MEDIUM

(71) Applicant: NEC Corporation, Minato-ku, Tokyo (JP)

(72) Inventor: Noriyuki TONOUCHI, Tokyo (JP)

(73) Assignee: NEC Corporation, Minato-ku, Tokyo (JP)

(21) Appl. No.: 18/709,863

(22) PCT Filed: Mar. 18, 2022
(86) PCT No.: PCT/JP2022/012707

§ 371 Date: May 14, 2024

(51) Int. Cl. G01N 33/00; G01N 1/44

(52) U.S. Cl. CPC G01N 33/0068; G01N 33/0031; G01N 1/44

(57) ABSTRACT

In order to enable highly accurate anomaly determination for a determination target, an anomaly determination apparatus (1) includes: an acquisition section (11) that acquires, from a plurality of gas sensors having different responsivities from each other depending on a composition of gas, measurement data for gas generated from a determination target; and a determination section (12) that determines an anomaly regarding the determination target by comparing reference data with determination data which is obtained from the measurement data from the respective plurality of gas sensors for the determination target and which is obtained in accordance with a relationship between the measurement data.

### TECHNICAL FIELD

The present invention relates to, for example, an anomaly determination apparatus that determines an anomaly in a determination target.

### BACKGROUND ART

There is a need for a technique for determining mixing of foreign matter into a sample. For example, Patent Literature 1 below discloses an electronic nose that is intended to carry out quality evaluation of a sample into which various stench components may be mixed. Specifically, the electronic nose disclosed in Patent Literature 1 uses a detection output from a plurality of odor sensors to determine whether an unknown sample serving as an object to be evaluated is a defective product.

### CITATION LIST

### Patent Literature

### Patent Literature 1

Japanese Patent Application Publication Tokukai No. 2003-232759

### SUMMARY OF INVENTION

### Technical Problem

The technique disclosed in Patent Literature 1 has a problem of making it impossible to carry out highly accurate determination.

An example aspect of the present invention has been made in view of such a problem, and an example object thereof is to provide, for example, an anomaly determination apparatus that enables highly accurate anomaly determination for a determination target.

### Solution to Problem

An anomaly determination apparatus according to an example aspect of the present invention includes: an acquisition means for acquiring, from a plurality of gas sensors having different responsivities from each other depending on a composition of gas, measurement data for gas generated from a determination target; and a determination means for determining an anomaly regarding the determination target by comparing reference data with determination data that is obtained from the measurement data from the respective plurality of gas sensors for the determination target and that is obtained in accordance with a relationship between the measurement data.

An anomaly determination method according to an example aspect of the present invention includes: acquiring, from a plurality of gas sensors having different responsivities from each other depending on a composition of gas, measurement data for gas generated from a determination target; and determining an anomaly regarding the determination target by comparing reference data with determination data that is obtained from the measurement data from the respective plurality of gas sensors for the determination target and that is obtained in accordance with a relationship between the measurement data.

An anomaly determination program according to an example aspect of the present invention causes a computer to function as: an acquisition means for acquiring, from a plurality of gas sensors having different responsivities from each other depending on a composition of gas, measurement data for gas generated from a determination target; and a determination means for determining an anomaly regarding the determination target by comparing reference data with determination data that is obtained from the measurement data from the respective plurality of gas sensors for the determination target and that is obtained in accordance with a relationship between the measurement data.

### Advantageous Effects of Invention

An example aspect of the present invention makes it possible to determine an anomaly regarding a determination target with high accuracy.

### BRIEF DESCRIPTION OF DRAWINGS

FIG. 1 is a block diagram illustrating a configuration of an anomaly determination apparatus according to a first example embodiment of the present invention.

FIG. 2 is a flowchart showing a flow of an example process of an anomaly determination method according to the first example embodiment of the present invention.

FIG. 3 is a block diagram illustrating a configuration of an anomaly determination apparatus according to a second example embodiment of the present invention.

FIG. 4 is a diagram illustrating an example gas measurement waveform obtained by a gas sensor according to the second example embodiment of the present invention.

FIG. 5 is a diagram illustrating example response waveforms of a plurality of gas sensors according to the second example embodiment of the present invention to a plurality of different measurement targets.

FIG. 6 is a diagram illustrating an example correspondence relationship between (a) an amplitude value of measurement data measured by the gas sensor according to the second example embodiment of the present invention and (b) a concentration of gas generated from a measurement target.

FIG. 7 is a diagram illustrating an example position of each sample in a set of samples according to the second example embodiment of the present invention.

FIG. 8 is a flowchart showing a flow of an example process of an anomaly detection method according to the second example embodiment of the present invention.

FIG. 9 is a flowchart showing a flow of an example process of a threshold determination method carried out by a threshold determination section according to the second example embodiment of the present invention.

FIG. 10 is a diagram illustrating an example configuration of a computer realizing a function as each of the apparatuses according to the respective example embodiments of the present invention.

### EXAMPLE EMBODIMENTS

### First Example Embodiment

The following description will discuss a first example embodiment of the present invention in detail with reference to the drawings. The present example embodiment is an embodiment serving as a basis for example embodiments described later.

### (Configuration of Anomaly Determination Apparatus)

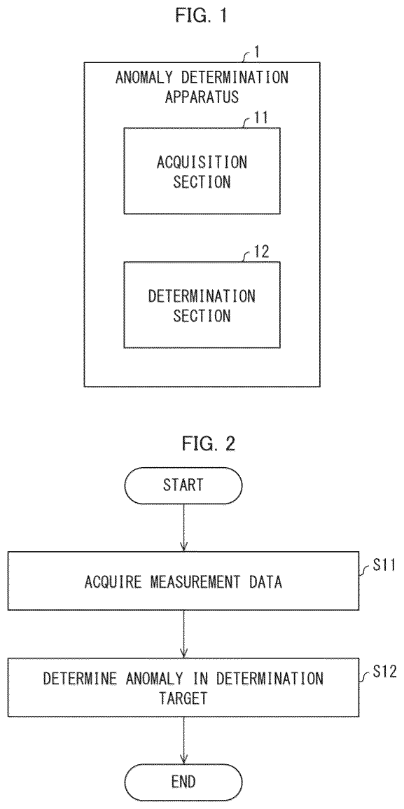

The following description will discuss a configuration of an anomaly determination apparatus 1 according to the present example embodiment with reference to FIG. 1. FIG. 1 is a block diagram illustrating the configuration of the anomaly determination apparatus 1. As illustrated in FIG. 1, the anomaly determination apparatus 1 includes an acquisition section (acquisition means) 11 and a determination section (determination means) 12.

The acquisition section 11 acquires, from a plurality of gas sensors having different responsivities from each other depending on a composition of gas, measurement data for gas generated from a determination target.

Note here that the gas sensors having different responsivities from each other are, for example, gas sensors by which measurement waveforms in different patterns are obtained in accordance with a composition of gas serving as a measurement target including the above-described determination target.

Note also that the measurement data is, for example, time series data, which is not intended to limit the present example embodiment.

The determination section 12 determines an anomaly regarding the determination target by comparing reference data with determination data. The determination data is obtained from the measurement data from the respective plurality of gas sensors for the determination target, and is obtained in accordance with a relationship between the measurement data.

Note here that the determination data obtained in accordance with a relationship between the measurement data refers to, for example, data obtained by using measurement data for the determination target, the measurement data being measured by a specific gas sensor among the plurality of gas sensors, to normalize each piece of the measurement data measured by the plurality of gas sensors.

Note also that the reference data may be, for example, data which is obtained from the measurement data from the respective plurality of gas sensors for the determination target that is normal and which is obtained in accordance with a relationship between the measurement data.

### (Effect of Anomaly Determination Apparatus)

As described above, in the anomaly determination apparatus 1 according to the present example embodiment, a configuration is employed such that determination data which is obtained in accordance with a relationship between measurement data from a plurality of gas sensors is used to determine an anomaly regarding a determination target. Thus, the anomaly determination apparatus 1 according to the present example embodiment brings about an effect of making it possible to determine an anomaly regarding a determination target with high accuracy.

### (Anomaly Determination Program)

The foregoing functions of the anomaly determination apparatus 1 can also be realized by a program. An estimation program according to the present example embodiment causes the anomaly determination apparatus 1 to function as: an acquisition means for acquiring, from a plurality of gas sensors having different responsivities from each other depending on a composition of gas, measurement data for gas generated from a determination target; and a determination means for determining an anomaly regarding the determination target by comparing reference data with determination data which is obtained from the measurement data from the respective plurality of gas sensors for the determination target and which is obtained in accordance with a relationship between the measurement data. The anomaly determination program makes it possible to determine an anomaly regarding a determination target with high accuracy.

### (Flow of Anomaly Determination Method)

The following description will discuss a flow of an anomaly determination method according to the present example embodiment with reference to FIG. 2. FIG. 2 is a flowchart showing the flow of the anomaly determination method. Note that steps of the anomaly determination method may be carried out by a processor of the anomaly determination apparatus 1.

In S11, at least one processor acquires, from a plurality of gas sensors having different responsivities from each other depending on a composition of gas, measurement data for gas generated from a determination target.

In S12, the at least one processor determines an anomaly regarding the determination target by comparing reference data with determination data. The determination data is obtained from the measurement data from the respective plurality of gas sensors for the determination target, and is obtained in accordance with a relationship between the measurement data.

### (Effect of Anomaly Determination Method)

As described above, in the anomaly determination method according to the present example embodiment, a configuration is employed such that determination data which is obtained in accordance with a relationship between measurement data from a plurality of gas sensors is used to determine an anomaly regarding a determination target. Thus, the anomaly determination method according to the present example embodiment brings about an effect of making it possible to determine an anomaly regarding a determination target with high accuracy.

### Second Example Embodiment

The following description will discuss a second example embodiment of the present invention in detail with reference to the drawings.

### (Configuration of Anomaly Detection Apparatus)

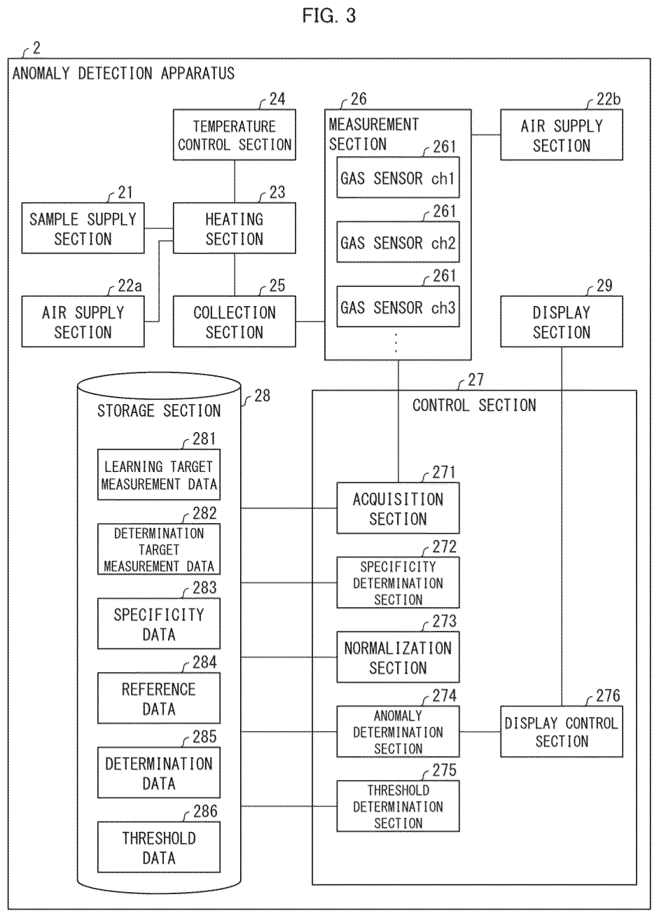

FIG. 3 is a block diagram illustrating a configuration of an anomaly detection apparatus 2 according to the present example embodiment. The anomaly detection apparatus 2 includes a sample supply section 21, an air supply section 22a, an air supply section 22b, a heating section (heating and burning section) 23, a temperature control section 24, a collection section 25, a measurement section 26, a control section (anomaly determination apparatus) 27, a storage section 28, and a display section 29.

### <Sample Supply Section 21>

The sample supply section 21 supplies a measurement sample to the heating section 23. Note that the sample supply section 21 need only be configured to be capable of supplying the measurement sample to the heating section 23, and a mechanism thereof is not particularly limited. Note here that the measurement sample includes, for example, a sample (determination target) which is to be subjected to anomaly determination (detection) carried out by the anomaly detection apparatus 2, and a sample which is normal. Note that the sample supplied to the heating section 23 need only be a substance which generates gas by burning by heating, and can be exemplified by a synthetic resin.

Note here that the sample which is normal is a sample in which no foreign matter is mixed. For example, the sample which is normal may be a sample whose material has a purity that is higher than a predetermined value. In the present specification, the sample which is normal is also referred to as a learning target.

<Air Supply Section 22a>

The air supply section 22a supplies air to the heating section 23. Note that the air supply section 22a need only be configured to be capable of supplying the air to the heating section 23, and a mechanism thereof is not particularly limited. The air supplied to the heating section 23 by the air supply section 22a is used to burn the measurement sample.

### <Heating Section 23>

The heating section 23 heats and burns the measurement sample so as to generate gas. For example, the heating section 23 heats and burns a determination target at a specific temperature so as to generate gas. The specific temperature in the heating section 23 is controlled by the temperature control section 24. Note that the heating section 23 need only be configured to be capable of heating the measurement sample, and a mechanism thereof is not particularly limited.

### <Temperature Control Section 24>

The temperature control section 24 controls the temperature at which the heating section 23 heats the measurement sample. For example, in a case where the measurement sample is a synthetic resin such as plastic, the temperature control section 24 carries out control so that the heating section 23 heats the measurement sample at 350° C. or higher. The temperature at which the heating section 23 heats the measurement sample may vary depending on the measurement sample. The temperature control section 24 controls the temperature, at which the heating section 23 heats the measurement sample, so that the temperature reaches a temperature at which the measurement sample burns.

### <Collection Section 25>

The collection section 25 collects gas generated from the measurement sample burned by heating by the heating section 23. The collection section 25 supplies the collected gas to the measurement section 26.

Note that, in the present example embodiment, the following description assumes that the collection section 25 is configured to collect gas which is generated by burning of the measurement sample. As another example, the collection section 25 may be configured to collect gas (e.g., a volatile substance or the like) that is generated without burning of the measurement sample.

### <Measurement Section 26>

The measurement section 26 includes a plurality of gas sensors 261 that carry out measurement with respect to gas supplied from the collection section 25. The plurality of gas sensors 261 have different responsivities from each other depending on a composition of gas. The plurality of gas sensors 261 may be configured, for example, as a gas sensor array. In the example illustrated in FIG. 3, the measurement section 26 includes a gas sensor ch1, a gas sensor ch2, a gas sensor ch3, and the like as the plurality of gas sensors 261. Each of the gas sensors 261 outputs, to an acquisition section 271 of the control section 27, a signal indicative of a measurement value.

<Air Supply Section 22b>

The air supply section 22b supplies air to the measurement section 26. Note that the air supply section 22b need only be configured to be capable of supplying the air to the measurement section 26, and a mechanism thereof is not particularly limited. The air supplied to the measurement section 26 by the air supply section 22b is used for the gas sensors 261 to carry out measurement with respect to gas.

(Method for Carrying Out Measurement with Respect to Gas)

The following description will discuss an example method in which the plurality of gas sensors 261 carry out measurement with respect to gas. The gas sensors 261 carry out measurement with respect to the gas in two modes, which are a sample gas supply mode and a clean air supply mode. The sample gas supply mode is a mode in which the plurality of gas sensors 261 carry out measurement with respect to the gas while the gas is being supplied from the collection section 25 to the measurement section 26. The clean air supply mode is a mode in which the plurality of gas sensors 261 carry out measurement with respect to the gas while the air is being supplied from the air supply section 22b to the measurement section 26. In the sample gas supply mode, the gas supplied from the collection section 25 is gradually filled in the measurement section 26. In the clean air supply mode, the gas that has been supplied from the collection section 25 and that is filled in the measurement section 26 is gradually exhausted from inside the measurement section 26.

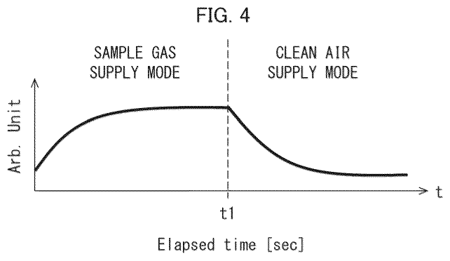

FIG. 4 is a diagram illustrating an example gas measurement waveform obtained by a gas sensor 261 according to the present example embodiment. A vertical axis in FIG. 4 shows “arbitrary unit (Arb. unit)”, which indicates a measurement value obtained by the gas sensor 261. A horizontal axis in FIG. 4 shows “elapsed time (sec)”. FIG. 4 illustrates an example gas measurement waveform in a case where gas is subjected to measurement in the sample gas supply mode until an elapsed time t1, and gas is subjected to measurement in the clean air supply mode from the elapsed time t1.

In the present specification, “measurement value” obtained by the gas sensor 261 may also be referred to as “measurement data” obtained by the gas sensor 261. Further, in the present specification, “measurement” by the gas sensor 261 may also be referred to as “response” by the gas sensor 261. Furthermore, in the present specification, “measurement waveform” of the gas sensor 261 may also be referred to as “response waveform” of the gas sensor 261.

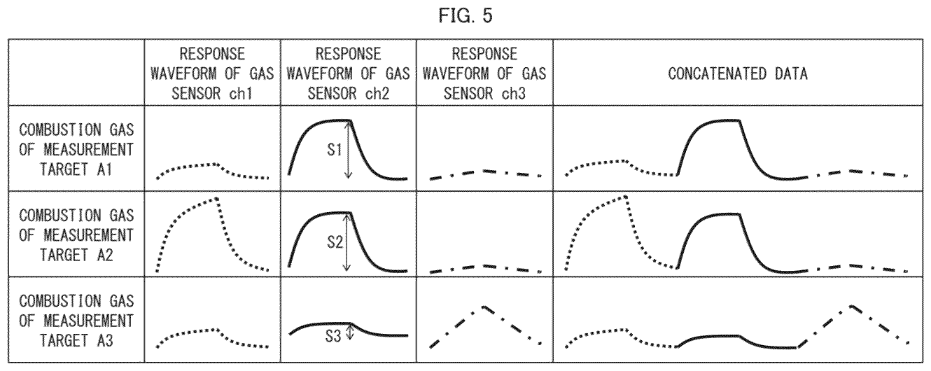

FIG. 5 is a diagram illustrating example response waveforms of the plurality of gas sensors 261 (gas sensors ch1, ch2, and ch3) according to the present example embodiment that have different responsivities from each other depending on a composition of gas to a plurality of different measurement targets (combustion gases of the measurement targets A, B, and C). As illustrated in FIG. 5, the response waveforms of the gas sensors ch1, ch2, and ch3 are response waveforms having different patterns depending on a composition of a measurement target.

### <Control Section 27>

The control section 27 includes an acquisition section (acquisition means) 271, a specificity determination section 272, a normalization section (normalization means) 273, an anomaly determination section (determination means) 274, a threshold determination section (threshold determination means) 275, and a display control section (display control means) 276 as illustrated in FIG. 3.

### <Acquisition Section 271>

The acquisition section 271 acquires, from the plurality of gas sensors 261 having different responsivities from each other depending on a composition of gas, measurement data for gas generated from a determination target.

Specifically, the acquisition section 271 acquires a signal indicative of a measurement value from each of the gas sensors 261 for the gas generated from the determination target. The acquisition section 271 uses the acquired measurement value to update determination target measurement data (measurement data) 282 stored in the storage section 28. The determination target measurement data 282 is data indicative of a measurement value obtained through measurement carried out by each of the gas sensors 261 (the gas sensor ch1, the gas sensor ch2, the gas sensor ch3, and the like) with respect to the gas generated from the determination target. For example, the determination target measurement data 282 may be data indicative of a response waveform of each of the gas sensors 261 to the determination target as illustrated in FIGS. 4 and 5. The determination target measurement data 282 may include data indicative of measurement values for a plurality of determination targets, the measurement values having been measured by the respective gas sensors 261.

Further, the acquisition section 271 may acquire a signal indicative of a measurement value from each of the gas sensors 261 for gas generated from a learning target. The acquisition section 271 uses the acquired measurement value to update learning target measurement data (measurement data) 281 stored in the storage section 28. The learning target measurement data 281 is data indicative of a measurement value obtained through measurement carried out by each of the gas sensors 261 (the gas sensor ch1, the gas sensor ch2, the gas sensor ch3, and the like) with respect to the gas generated from the learning target. For example, the determination target measurement data 282 may be data indicative of a response waveform of each of the gas sensors 261 to the learning target as illustrated in FIGS. 4 and 5. Note that the learning target measurement data 281 need only be a measurement value from each of the gas sensors 261 for gas generated from a sample which is normal. For example, measurement data measured by an external device including a gas sensor similar to the gas sensors 261 of the measurement section 26 may be applied.

The acquisition section 271 that has updated the determination target measurement data 282 or the learning target measurement data 281 stored in the storage section 28 outputs, to the specificity determination section 272, a signal indicative of such update.

### <Specificity Determination Section 272>

The specificity determination section 272 that has received, from the acquisition section 271, a signal indicative of update of the determination target measurement data 282 or the learning target measurement data 281 determines specificity of the gas sensors 261. Specifically, the specificity determination section 272 determines whether a gas sensor 261 has lower specificity than a predetermined criterion to a plurality of measurement targets which have been subjected to measurement by the gas sensors 261 (gas sensors ch1, ch2, ch3, etc.).

Note here that “has low specificity” can also be expressed as having low selectivity for a plurality of measurement targets which have been subjected to measurement. Alternatively, “has low specificity” can be expressed as having a high responsivity to a plurality of measurement targets which have been subjected to measurement. For example, a gas sensor having low specificity is a gas sensor that responds to any measurement target which has been subjected to measurement.

### (Method for Determining Specificity of Gas Sensor)

The following description will discuss an example method in which the specificity determination section 272 determines specificity of each of the gas sensors 261.

In the present example, in a case where an amplitude value of measurement data for all gasses (measurement targets), the measurement data being measured by a certain gas sensor 261, is greater than a predetermined value, the specificity determination section 272 determines that the certain gas sensor 261 has low specificity.

For example, in a case where an amplitude value of measurement data for all the measurement targets, the measurement data being measured by the gas sensor ch1, is greater than a value 10 times a standard deviation of the amplitude value of the measurement data from the gas sensor ch1 in a blank, it is determined that specificity of the gas sensor ch1 is lower than the predetermined criterion. Note here that the blank refers to a state in which there is no measurement target, for example, a state in which only air is present. The specificity determination section 272 may use, as the measurement data in the blank, measurement data obtained during measurement with respect to noise. Note also that the above numerical value “10 times” can be changed as appropriate.

The specificity determination section 272 may use, as the above-described “measurement data for gas (measurement target), the measurement data having been measured by each of the gas sensors 261”, at least one selected from the group consisting of data indicated by the learning target measurement data 281 and data indicated by the determination target measurement data 282. Alternatively, the specificity determination section 272 may use, as the above-described “measurement data for gas (measurement target), the measurement data having been measured by each of the gas sensors 261′, all data indicated by the learning target measurement data 281 and the determination target measurement data 282. The specificity determination section 272 may determine specificity of each of the gas sensors 261 every time the learning target measurement data 281 and the determination target measurement data 282 are updated.

The specificity determination section 272 may determine that a gas sensor 261 for which the following expression holds has lower specificity than the predetermined criterion.

$${Amp}_{j} ( k , {g}_{i} ) > threshold * \sqrt{\frac{1}{m} {\sum }_{j = 1}^{m} {\{ {Amp}_{j} ( k , none ) - \overline{Amp ( k , none )} \}}^{2}}$$

$$where$$

$${g}_{i} ( i : 0 ∼ n )$$

represents a gas (measurement target) having been subjected to measurement by a gas sensor k.

Further,
Ampⱼ(k, gᵢ)
represents an amplitude value of measurement data from the gas sensor k for the gas gᵢ.

Furthermore, the gas sensor k carries out measurements a plurality of times with respect to the gas gᵢ, and j represents a measurement number for distinguishing a specific measurement from the measurements carried out the plurality of times with respect to the gas gᵢ.

Further, a standard deviation of
Amp(k, gᵢ)
in a case where the gas sensor k carries out measurements m times with respect to the gas gᵢ can be represented by

$$\sqrt{\frac{1}{m} {\sum }_{j = 1}^{m} {\{ {Amp}_{j} ( k , {g}_{i} ) - \overline{Amp ( k , {g}_{i} )} \}}^{2}} .$$

In other words, the specificity determination section 272 determines that the gas sensor k for which the following expression holds has lower specificity than the predetermined criterion.

Amplitude value of measurement data from gas sensor k for gas gᵢ>threshold*standard deviation of amplitude value of measurement data from gas sensor k in blank

Furthermore, the specificity determination section 272 may further select a gas sensor 261 from among gas sensors 261 that have been determined to have lower specificity than the predetermined criterion. For example, the specificity determination section 272 may select a gas sensor 261 whose amplitude value of measurement data for all the measurement targets, the measurement data having been measured by the gas sensor 261, is in a predetermined range. The above “predetermined range” can be exemplified by a range in which a relationship between an amplitude value and a concentration of gas generated from a measurement target is linear.

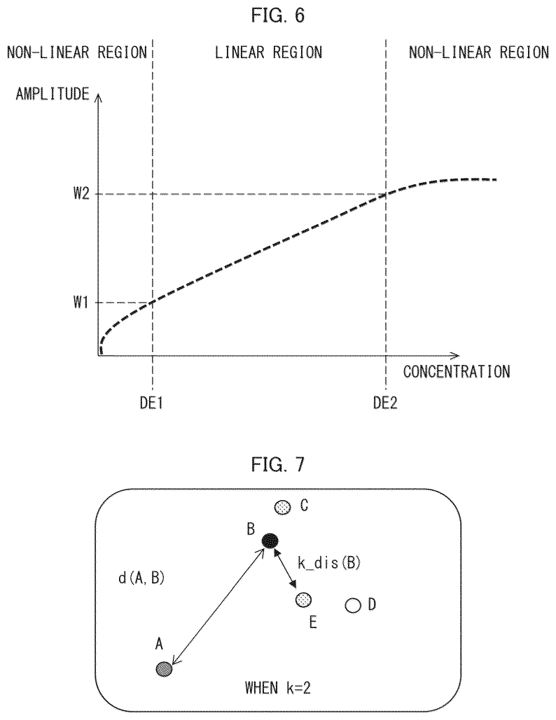

The following description will use FIG. 6 to discuss a correspondence relationship between (a) an amplitude value of measurement data measured by a gas sensor 261 and (b) a concentration of gas generated from a measurement target. FIG. 6 is a diagram illustrating an example correspondence relationship between (a) an amplitude value of measurement data measured by the gas sensor 261 and (b) a concentration of gas generated from a measurement target. A vertical axis of a graph illustrated in FIG. 6 shows the amplitude value of the measurement data measured by the gas sensor 261. A horizontal axis of the graph illustrated in FIG. 6 shows the concentration of the gas generated from the measurement target. As illustrated in FIG. 6, the relationship between the amplitude value and the concentration of the gas is non-linear in a region in which the amplitude value is smaller than W1 and a region in which the amplitude value is greater than W2. Further, the relationship between the amplitude value and the concentration of the gas is linear in a region in which the amplitude value is in a range from W1 to W2.

In the example illustrated in FIG. 6, the specificity determination section 272 selects a gas sensor 261 whose amplitude value of measurement data for all measurement targets, the measurement data having been measured by the gas sensor 261, is in the range from W1 to W2.

The specificity determination section 272 updates, in accordance with the above-described determination result and selection, specificity data 283 stored in the storage section 28. The specificity data 283 is data indicative of a gas sensor 261 that has lower specificity than the predetermined criterion.

The specificity determination section 272 may combine the above-described “method for determining specificity of a gas sensor” and “selection of a gas sensor” to select only one gas sensor 261 that is determined to have lower specificity than the predetermined criterion.

In a case where the above-described “method for determining specificity of a gas sensor” and “selection of a gas sensor” are combined but there are a plurality of gas sensors 261 that are determined to have lower specificity than the predetermined criterion, the specificity determination section 272 may carry out the following additional selection process with respect to the plurality of gas sensors 261.

For example, the specificity determination section 272 may change, as appropriate, a value of the “threshold” in Amplitude value of measurement data from gas sensor k for gas gᵢ>threshold*standard deviation of amplitude value of measurement data from gas sensor k in blank described in the above-described “method for determining specificity of a gas sensor”, and repeatedly carry out selection until only one gas sensor 261 that has lower specificity than the predetermined criterion is obtained.

Further, the specificity determination section 272 may calculate a correlation coefficient between the respective gas sensors that is defined by the following equation, and select a sensor in which a sum of correlation coefficients reaches a maximum.

$$corr ( k , {k}^{′} , {g}_{i} ) = \frac{{\sum }_{j = 1}^{m} ( {Amp}_{j} ( k , {g}_{i} ) - \overline{Amp ( k , {g}_{i} )} ) ( {Amp}_{j} ( {k}^{′} , {g}_{i} ) - \overline{Amp ( {k}^{′} , {g}_{i} )} )}{\begin{matrix}\sqrt{{\sum }_{j = 1}^{m} {\{ {Amp}_{j} ( k , {g}_{i} ) - \overline{Amp ( k , {g}_{i} )} \}}^{2}} \\ \sqrt{{\sum }_{j = 1}^{m} {\{ {Amp}_{j} ( {k}^{′} , {g}_{i} ) - \overline{Amp ( {k}^{′} , {g}_{i} )} \}}^{2}}\end{matrix}}$$

The above equation represents a correlation coefficient between the gas sensor k and a gas sensor k′ in a case where the gas sensor k and the gas sensor k′ carry out measurements m times with respect to the gas gᵢ.

The specificity determination section 272 that has completed determination of specificity of a gas sensor 261 outputs, to the normalization section 273, a signal indicative of completion of the determination.

### <Normalization Section 273>

The normalization section 273 that has received a signal indicative of completion of the determination from the specificity determination section 272 generates determination data 285 and reference data 284.

The normalization section 273 generates the determination data 285 by normalizing the determination target measurement data 282 measured by the plurality of gas sensors 261. The normalization section 273 carries out the above-described normalization with use of an amplitude value of measurement data for the determination target, the measurement data being measured by a gas sensor that is among the plurality of gas sensors 261 and that has lower specificity than the predetermined criterion to a plurality of measurement targets which have been subjected to measurement by the plurality of gas sensors 261.

Different measurement targets composed of an identical composition are identical in composition of gases generated from the respective measurement targets. However, in a case where the gases generated from the respective measurement targets have different concentrations, a gas sensor 261 will measure different measurement values in accordance with the concentrations of the gases generated by the respective measurement targets. In order to prevent the concentrations of the gasses generated from the respective measurement targets from affecting measurement, the normalization section 273 generates the determination data 285 by normalizing a measurement value measured by the gas sensor 261.

The following description will discuss, with reference to FIG. 5, normalization carried out by the normalization section 273. The normalization section 273 refers to the specificity data 283 stored in the storage section 28, and determines a gas sensor that has lower specificity than the predetermined criterion. In the example illustrated in FIG. 5, the gas sensor ch2 is a gas sensor having lower specificity than the predetermined criterion. The normalization section 273 carries out the normalization by using an amplitude value (amplitude maximum value) of a measurement value measured by the gas sensor ch2 to divide measurement values measured by the gas sensors 261 (the gas sensor ch1, the gas sensor ch2, the gas sensor ch3).

For example, as illustrated in FIG. 5, the amplitude value of the measurement value measured by the gas sensor ch2 for the combustion gas of the measurement target A1 is S1. The normalization section 273 carries out the normalization by dividing, by S1, the measurement values measured by the gas sensor ch1, the gas sensor ch2, and the gas sensor ch3 for the combustion gas of the measurement target A1.

Further, the amplitude value of the measurement value measured by the gas sensor ch2 for the combustion gas of the measurement target A2 is S2. The normalization section 273 carries out the normalization by dividing, by S2, the measurement values measured by the gas sensor ch1, the gas sensor ch2, and the gas sensor ch3 for the combustion gas of the measurement target A2.

Furthermore, the amplitude value of the measurement value measured by the gas sensor ch2 for the combustion gas of the measurement target A3 is S3. The normalization section 273 carries out the normalization by dividing, by S3, the measurement values measured by the gas sensor ch1, the gas sensor ch2, and the gas sensor ch3 for the combustion gas of the measurement target A2.

The normalization section 273 stores, in the storage section 28, the determination data 285 generated by normalization.

Further, the normalization section 273 generates the reference data 284 by normalizing the learning target measurement data 281, which is measurement data for the learning target, the measurement data being measured by the plurality of gas sensors 261. The normalization section 273 carries out the above-described normalization with use of an amplitude value of measurement data for the learning target, the measurement data being measured by a gas sensor that is among the plurality of gas sensors 261 and that has lower specificity than the predetermined criterion to a plurality of measurement targets which have been subjected to measurement by the plurality of gas sensors 261.

Note here that the reference data is data that is obtained in accordance with a relationship between measurement data from a plurality of gas sensors for gas generated from a learning target which is normal.

Since generation of the reference data 284 by the normalization section 273 is similar to generation of the determination data 285 described earlier, a description thereof is omitted here. The normalization section 273 stores the generated reference data 284 in the storage section 28.

The normalization section 273 that has generated the determination data 285 and the reference data 284 outputs, to the anomaly determination section 274, a signal indicative of such generation.

### <Anomaly Determination Section 274>

The anomaly determination section 274 that has received, from the normalization section 273, the signal indicative of generation of the determination data 285 and the reference data 284 determines an anomaly regarding the determination target.

Specifically, the anomaly determination section 274 determines an anomaly regarding the determination target by comparing reference data with determination data that is obtained from the measurement data from the respective plurality of gas sensors 261 for the determination target and that is obtained in accordance with a relationship between the measurement data.

### (Anomaly Determination Method: Local Outlier Factor Method)

The following description will discuss an example anomaly determination method carried out by the anomaly determination section 274. In the present example, the anomaly determination section 274 uses a local outlier factor method to determine an anomaly regarding the determination target.

In anomaly determination in the present example, one-dimensional concatenated data is used that is generated with use of the measurement values (response waveforms) measured by all the gas sensors (the gas sensor ch1, the gas sensor ch2, and the gas sensor ch3) of the measurement section 26 as illustrated in FIG. 5. Note that the one-dimensional connection data is connection data obtained by serially concatenating, in series, the measurement values measured by the respective gas sensors (the gas sensor ch1, the gas sensor ch2, and the gas sensor ch3) and normalized by the normalization section 273.

For example, the one-dimensional concatenated data can be obtained by concatenating the response waveform of the gas sensor ch1, the response waveform of the gas sensor ch2, and the response waveform of the gas sensor ch3 in series in this order.

In the description of the present anomaly determination method, one piece of one-dimensional data is referred to as a sample. Further, in the description of the present anomaly determination method, it is assumed that a sample A is a sample based on the determination data 285 and that an anomaly regarding the sample A is determined. That is, the sample A can be expressed as determination data. Furthermore, a sample other than the sample A is a sample based on the reference data 284, and the sample other than the sample A can be expressed as reference data.

It is assumed that k_dis (A) is a distance to a kth neighbor of the sample A and that and Nₖ (A) is a set of k nearest neighbor samples. It is also assumed that d (A, B) is a distance between the sample A and a sample B. The anomaly determination section 274 calculates the following reachability distance and local reachability density.

Reachability distance:
RDₖ(A,B)=max{k_dis(B), d(A,B)}

Local reachability density:

$${lrd}_{k} ( A ) = 1 / ( \frac{{\sum }_{B ∈ {N}_{k} ( A )} R {D}_{k} ( A , B )}{\vert {N}_{k} ( A ) \vert} )$$

FIG. 7 is a diagram illustrating an example position of each sample in a set of samples. For example, as illustrated in FIG. 7, when a value of k is 2, the local reachability density is represented by the following expression.

$$1 / ( \frac{R {D}_{2} ( A , B ) + R {D}_{2} ( A , E )}{2} )$$

The anomaly determination section 274 calculates LOF_{K}(A) by dividing an average of local reachability densities of a neighbor group by a local reachability density of the sample A, as represented by the following equation.

$${LOF}_{k} ( A ) = \frac{{\sum }_{B ∈ {N}_{k} ( A )} lrd ( B )}{\vert {N}_{k} ( A ) \vert} / lr d ( A )$$

When LOF_{K} (A) has a value close to 1, the sample A is located at a density substantially equal to that near the sample A. When LOF_{K} (A) has a value below 1, the sample A is located in a dense region. LOF_{K} (A) that has a value sufficiently greater than 1 means that the sample A is an outlier.

Further, the anomaly determination section 274 may determine an anomaly regarding the determination target with use of a local outlier factor method in which a later-described threshold determined by the threshold determination section 275 is used. For example, in a case where the calculated LOF_{K}(A) is greater than the threshold determined by the threshold determination section 275, the anomaly determination section 274 determines that there is an anomaly in the sample A.

In the above-described anomaly determination method, it is possible to express that a distance between samples is defined for each time slice of measurement data, and accumulation of the distance is used to define LOF.

The anomaly determination section 274 which has determined that there is an anomaly in the determination target outputs, to the display control section, a signal indicating that there is an anomaly in the determination target.

### <Threshold Determination Section 275>

The threshold determination section 275 determines, from a distribution of the reference data, a threshold used for determination of an anomaly regarding the determination target by the anomaly determination section 274 with use of the local outlier factor method. For example, the threshold determination section 275 may determine an appropriate threshold from an LOF value distribution of the reference data 284. The threshold determination section 275 stores, in the storage section 28, threshold data 286 that indicates the threshold thus set.

(Anomaly Determination Method: Method with Use of at Least One of Principal Component Analysis and Neural Network)

The anomaly determination method carried out by the anomaly determination section 274 is not limited to the above-described example. For example, the anomaly determination section 274 may be configured to determine an anomaly with use of at least one of (i) a principal component analysis (PCA) in which the determination data 285 generated by the normalization section 273 by normalization is used and (ii) a neural network in which the determination data 285 is used as input data.

### <Display Control Section 276>

In a case where the anomaly determination section 274 determines that there is an anomaly in the determination target, the display control section 276 causes the display section 29 to display an indication indicating that there is an anomaly in the determination target.

### <Storage Section 28>

The storage section 28 stores the learning target measurement data 281, the determination target measurement data 282, the specificity data 283, the reference data 284, the determination data 285, and the threshold data 286.

### <Display Section 29>

The display section 29 displays an indication indicating that there is an anomaly in the determination target. The present example embodiment has discussed a configuration in which the anomaly detection apparatus 2 displays, in the display section 29, the fact that there is an anomaly in the determination target. Alternatively, for example, a configuration may be employed such that the anomaly detection apparatus 2 outputs, in audio form from an audio output section or the like, the fact that there is an anomaly in the determination target. A configuration in which the anomaly detection apparatus 2 notifies a user that there is an anomaly in the determination target is not particularly limited.

### (Flow of Anomaly Detection Method Carried Out by Anomaly Detection Apparatus 2)

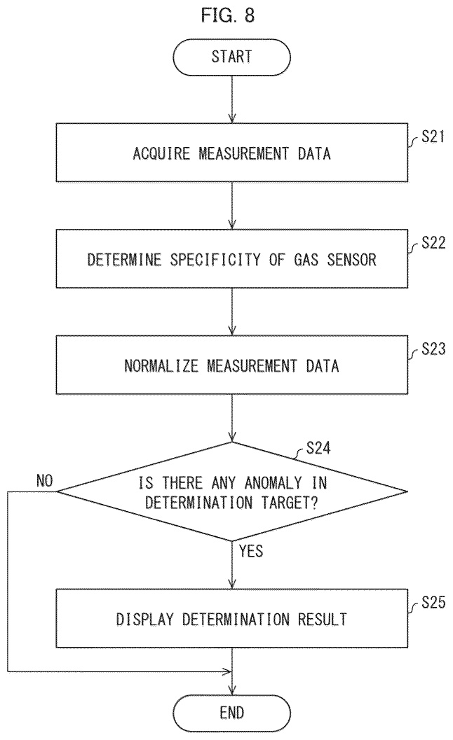

The following description will discuss, with reference to FIG. 8, a flow of a process of an anomaly determination method carried out by the anomaly detection apparatus 2 according to the present example embodiment. FIG. 8 is a flowchart showing a flow of an example process of the anomaly detection method carried out by the anomaly detection apparatus 2.

In S21, the acquisition section 271 acquires, from the plurality of gas sensors 261 (the gas sensor ch1, the gas sensor ch2, the gas sensor ch3, and the like) having different responsivities from each other depending on a composition of gas, measurement data for gas generated from a determination target. The acquisition section 271 updates the determination target measurement data 282 stored in the storage section 28.

In S22, the specificity determination section 272 determines specificity of the gas sensors 261 (the gas sensor ch1, the gas sensor ch2, the gas sensor ch3, and the like). Specifically, the specificity determination section 272 determines whether a gas sensor 261 has lower specificity than a predetermined criterion to a plurality of measurement targets which have been subjected to measurement by the gas sensors 261 (the gas sensor ch1, the gas sensor ch2, the gas sensor ch3, and the like).

After S22, in S23, the normalization section 273 generates the determination data 285 by normalizing the determination target measurement data 282. Further, the normalization section 273 generates the reference data 284 by normalizing the learning target measurement data 281, which is measurement data for the learning target. Specifically, the normalization section 273 carries out the above-described normalization with use of an amplitude value of measurement data measured by a gas sensor that is among the plurality of gas sensors 261 and that has lower specificity than the predetermined criterion to a plurality of measurement targets which have been subjected to measurement by the plurality of gas sensors 261.

After S23, in S24, the anomaly determination section 274 determines an anomaly regarding the determination target by comparing the reference data 284 with the determination data 285. The determination data 285 is obtained from the measurement data from the respective plurality of gas sensors 261 (the gas sensor ch1, the gas sensor ch2, the gas sensor ch3, and the like) for the determination target. Further, the determination data 285 is obtained in accordance with a relationship between the measurement data. Note that “obtained in accordance with a relationship between the measurement data” means, for example, that in S23, the normalization section 273 generates the determination data 285 by normalizing the determination target measurement data 282. In a case where the anomaly determination section 274 determines that there is an anomaly in the determination target (YES in S24), the process proceeds to S25. In a case where the anomaly determination section 274 determines that there is no anomaly in the determination target (NO in S24), the process ends.

In S25, the display control section 276 causes the display section 29 to display an indication indicating that there is an anomaly in the determination target. Then, the process ends. Note that the anomaly determination section 274 which has determined that there is no anomaly in the determination target (NO in S24) may be configured to output, to the display control section 276, a signal indicating that there is no anomaly in the determination target. In such a configuration, the display control section 276 may cause the display section 29 to display an indication indicating that there is no anomaly in the determination target.

### (Flow of Threshold Determination Method Carried Out by Anomaly Detection Apparatus 2)

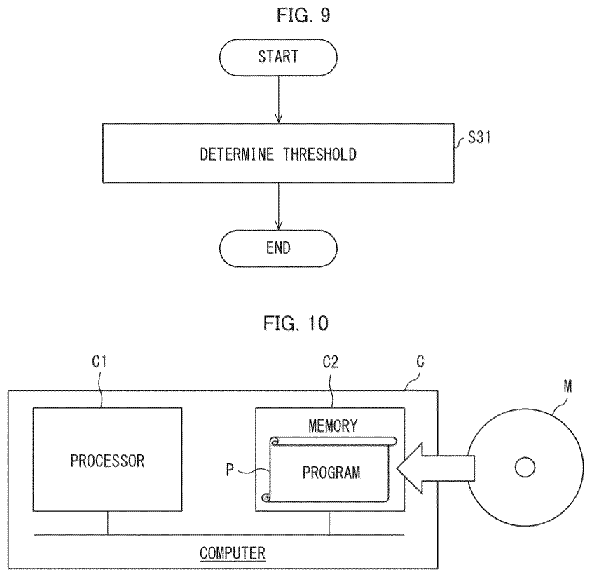

The following description will discuss, with reference to FIG. 9, a flow of a process of a threshold determination method carried out by the anomaly detection apparatus 2 according to the present example embodiment. FIG. 9 is a flowchart showing a flow of an example process of the threshold determination method carried out by the threshold determination section 275 of the anomaly detection apparatus 2.

In S31, the threshold determination section 275 determines a threshold from a distribution of measurement data for the learning target, the measurement data having been normalized and being shown in the reference data 284. For example, the threshold determination section 275 may determine the threshold after the process in which the reference data 284 is generated (the measurement data for the learning target is normalized) by the normalization section 273. Alternatively, after the threshold determination section 275 determines the threshold, the anomaly determination section 274 may use the threshold to determine an anomaly regarding the determination target.

### (Effect of Anomaly Detection Apparatus)

As described above, in the anomaly detection apparatus 2 according to the present example embodiment, the normalization section 273 is employed which generates the preceding determination data by using an amplitude value of measurement data for the determination target, the measurement data being measured by a gas sensor that is among the plurality of gas sensors 261 and that has lower specificity than a predetermined criterion to a plurality of measurement targets which have been subjected to measurement by the plurality of gas sensors 261, to normalize the measurement data measured by the plurality of gas sensors. Thus, the anomaly detection apparatus 2 according to the present example embodiment brings about not only the effect brought about by the anomaly determination apparatus 1 according to the first example embodiment but also an effect of making it possible to carry out anomaly determination with high accuracy while preventing a concentration of gas generated from a determination target from affecting the anomaly determination.

Further, in the anomaly detection apparatus 2 according to the present example embodiment, the reference data 284 is data that is obtained in accordance with a relationship between measurement data from the plurality of gas sensors 261 for gas generated from a learning target which is normal, and the normalization section 273 is employed which generates the reference data 284 by using an amplitude value of measurement data for the learning target, the measurement data being measured by a gas sensor that is among the plurality of gas sensors 261 and that has lower specificity than a predetermined criterion to a plurality of measurement targets which have been subjected to measurement by the plurality of gas sensors 261, to normalize the measurement data measured by the plurality of gas sensors 261 for the learning target. Thus, the anomaly detection apparatus 2 according to the present example embodiment brings about not only the effect brought about by the anomaly determination apparatus 1 according to the first example embodiment but also an effect of making it possible to carry out anomaly determination with high accuracy while preventing a concentration of gas generated from a learning target from affecting the anomaly determination.

Furthermore, in the anomaly detection apparatus 2 according to the present example embodiment, the anomaly determination section 274 is employed which uses a local outlier factor method to determine an anomaly regarding a determination target. Thus, the anomaly detection apparatus 2 according to the present example embodiment brings about not only the effect brought about by the anomaly determination apparatus 1 according to the first example embodiment but also an effect of making it possible to carry out anomaly determination with high accuracy with use of a local outlier factor method.

Further, in the anomaly detection apparatus 2 according to the present example embodiment, the reference data 284 is data that is obtained in accordance with a relationship between measurement data from the plurality of gas sensors 261 for gas generated from a learning target which is normal, and the threshold determination section 275 is employed which determines a threshold from a distribution of the reference data 284. Thus, the anomaly detection apparatus 2 according to the present example embodiment brings about not only the effect brought about by the anomaly determination apparatus 1 according to the first example embodiment but also an effect of making it possible to determine an anomaly with high accuracy with use of a local outlier factor method to which a threshold determined from a distribution of reference data is applied.

Furthermore, a configuration is employed such that the anomaly detection apparatus 2 according to the present example embodiment includes: the heating section 23 that heats and burns a determination target so as to generate gas; the plurality of gas sensors 261 that measure gas and that have different responsivities from each other depending on a composition of gas; the display section 29; and the control section 27 that further includes the display control section 276 which, in a case where it is determined that there is an anomaly in the determination target, causes the display section 29 to display an indication indicating that there is an anomaly in the determination target. Thus, the anomaly detection apparatus 2 according to the present example embodiment brings about an effect similar to the effect brought about by the anomaly determination apparatus 1 according to the first example embodiment.

### [Software Implementation Example]

Some or all of the functions of each of the anomaly determination apparatus 1 and the anomaly detection apparatus 2 may be realized by hardware such as an integrated circuit (IC chip) or may be alternatively realized by software.

In the latter case, the anomaly determination apparatus 1 and the anomaly detection apparatus 2 are each realized by, for example, a computer that executes instructions of a program that is software realizing the functions. FIG. 10 illustrates an example of such a computer (hereinafter, referred to as “computer C”). The computer C includes at least one processor C1 and at least one memory C2. The memory C2 stores a program P for causing the computer C to operate as each of the anomaly determination apparatus 1 and the anomaly detection apparatus 2. In the computer C, the functions of each of the anomaly determination apparatus 1 and the anomaly detection apparatus 2 are realized by the processor C1 reading the program P from the memory C2 and executing the program P.

The processor C1 can be, for example, a central processing unit (CPU), a graphic processing unit (GPU), a digital signal processor (DSP), a micro processing unit (MPU), a floating point number processing unit (FPU), a physics processing unit (PPU), a microcontroller, or a combination thereof. The memory C2 can be, for example, a flash memory, a hard disk drive (HDD), a solid state drive (SSD), or a combination thereof.

Note that the computer C may further include a random access memory (RAM) in which the program P is loaded when executed and/or in which various kinds of data are temporarily stored. The computer C may further include a communication interface for transmitting and receiving data to and from another apparatus. The computer C may further include an input/output interface through which the computer C is to be connected to an input/output device(s) such as a keyboard, a mouse, a display and/or a printer.

The program P can also be recorded in a non-transitory tangible storage medium M from which the computer C can read the program P. The storage medium M can be, for example, a tape, a disk, a card, a semiconductor memory, a programmable logic circuit, or the like. The computer C can acquire the program P via the storage medium M. The program P can be transmitted via a transmission medium. The transmission medium can be, for example, a communication network, a broadcast wave, or the like. The computer C can acquire the program P also via the transmission medium.

### [Additional Remark 1]

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### [Additional Remark 2]

The whole or part of the example embodiments disclosed above can also be described as below. Note, however, that the present invention is not limited to the following supplementary notes.

### (Supplementary Note 1)

An anomaly determination apparatus including:
an acquisition means for acquiring, from a plurality of gas sensors having different responsivities from each other depending on a composition of gas, measurement data for gas generated from a determination target; and
a determination means for determining an anomaly regarding the determination target by comparing reference data with determination data that is obtained from the measurement data from the respective plurality of gas sensors for the determination target and that is obtained in accordance with a relationship between the measurement data.

According to the above configuration, determination data that is obtained in accordance with a relationship between measurement data is used to determine an anomaly. This makes it possible to determine an anomaly with high accuracy.

### (Supplementary Note 2)

The anomaly determination apparatus according to Supplementary note 1, further including a normalization means,
the normalization means generating the preceding determination data by using an amplitude value of measurement data for the determination target, the measurement data being measured by a gas sensor that is among the plurality of gas sensors and that has lower specificity than a predetermined criterion to a plurality of measurement targets which have been subjected to measurement by the plurality of gas sensors, to normalize the measurement data measured by the plurality of gas sensors.

According to the above configuration, the determination data is normalized. This makes it possible to carry out anomaly determination with high accuracy while preventing a concentration of gas generated from a determination target from affecting the anomaly determination.

### (Supplementary Note 3)

The anomaly determination apparatus according to Supplementary note 1 or 2, further including a normalization means,
the reference data being data that is obtained in accordance with a relationship between measurement data from the plurality of gas sensors for gas generated from a learning target which is normal, and
the normalization means generating the preceding reference data by using an amplitude value of measurement data for the learning target, the measurement data being measured by a gas sensor that is among the plurality of gas sensors and that has lower specificity than a predetermined criterion to a plurality of measurement targets which have been subjected to measurement by the plurality of gas sensors, to normalize the measurement data measured by the plurality of gas sensors for the learning target.

According to the above configuration, the reference data is normalized. This brings about an effect of making it possible to carry out anomaly determination with high accuracy while preventing a concentration of gas generated from a learning target from affecting the anomaly determination.

### (Supplementary Note 4)

The anomaly determination apparatus according to any one of Supplementary notes 1 to 3, wherein
the determination means uses a local outlier factor method to determine an anomaly regarding the determination target.

The above configuration makes it possible to use a local outlier factor method to determine an anomaly with high accuracy.

### (Supplementary Note 5)

The anomaly determination apparatus according to Supplementary note 4, wherein
the reference data is data that is obtained in accordance with a relationship between measurement data from the plurality of gas sensors for gas generated from a learning target which is normal,
the anomaly determination apparatus further including a threshold determination means for determining a threshold from a distribution of the reference data,
the determination means determining the anomaly regarding the determination target with use of the local outlier factor method in which the threshold is used.

The above configuration brings about an effect of making it possible to determine an anomaly with high accuracy with use of a local outlier factor method in which a threshold determined from a distribution of reference data is applied.

### (Supplementary Note 6)

An anomaly detection apparatus including:
a heating and burning section that heats and burns a determination target so as to generate gas;
a plurality of gas sensors that carry out measurement with respect to the gas and that have different responsivities from each other depending on a composition of gas;
a display section; and
an anomaly determination apparatus according to any one of Supplementary notes 1 to 5, the anomaly determination apparatus further including a display control means for, in a case where it is determined that there is an anomaly in the determination target, causing the display section to display an indication indicating that there is an anomaly in the determination target.

The above configuration brings about an effect similar to that brought about by the above-described anomaly determination apparatus.

### (Supplementary Note 7)

An anomaly determination method including:
acquiring, from a plurality of gas sensors having different responsivities from each other depending on a composition of gas, measurement data for gas generated from a determination target; and
determining an anomaly regarding the determination target by comparing reference data with determination data that is obtained from the measurement data from the respective plurality of gas sensors for the determination target and that is obtained in accordance with a relationship between the measurement data.

The above method brings about an effect similar to that brought about by the above-described anomaly determination apparatus.

### (Supplementary Note 8)

An anomaly determination program for causing a computer to function as:
an acquisition means for acquiring, from a plurality of gas sensors having different responsivities from each other depending on a composition of gas, measurement data for gas generated from a determination target; and
a determination means for determining an anomaly regarding the determination target by comparing reference data with determination data that is obtained from the measurement data from the respective plurality of gas sensors for the determination target and that is obtained in accordance with a relationship between the measurement data.

The above configuration brings about an effect similar to that brought about by the above-described anomaly determination apparatus.

### [Additional Remark 3]

The whole or part of the example embodiments disclosed above further can also be expressed as follows.

An anomaly determination apparatus including at least one processor, the at least one processor carrying out: an acquisition process for acquiring, from a plurality of gas sensors having different responsivities from each other depending on a composition of gas, measurement data for gas generated from a determination target; and a determination process for determining an anomaly regarding the determination target by comparing reference data with determination data that is obtained from the measurement data from the respective plurality of gas sensors for the determination target and that is obtained in accordance with a relationship between the measurement data.

Note that the anomaly determination apparatus may further include a memory, which may store a program for causing the at least one processor to carry out the acquisition process and the determination process. Further, the program may be stored in a non-transitory tangible computer-readable storage medium.

### REFERENCE SIGNS LIST

1 . . . Anomaly determination apparatus
11, 271 . . . Acquisition section (acquisition means)
12 . . . Determination section (determination means)
23 . . . Heating section (heating and burning section)
261, k . . . Gas sensor
27 . . . Control section (anomaly determination apparatus)
273 . . . Normalization section (normalization means)
274 . . . Anomaly determination section (determination means)
275 . . . Threshold determination section (threshold determination means)
276 . . . Display control section (display control means)
281 . . . Learning target measurement data (measurement data)
282 . . . Determination target measurement data (measurement data)
285 . . . Determination data
284 . . . Reference data
29 . . . Display section

What is claimed is:

1. An anomaly determination apparatus comprising at least one processor, the at least one processor carrying out:
an acquisition process for acquiring, from a plurality of gas sensors having different responsivities from each other depending on a composition of gas, measurement data for gas generated from a determination target; and
a determination process for determining an anomaly regarding the determination target by comparing reference data with determination data that is obtained from the measurement data from the respective plurality of gas sensors for the determination target and that is obtained in accordance with a relationship between the measurement data.

2. The anomaly determination apparatus according to claim 1, wherein the at least one processor further carries out a normalization process,
in the normalization process, the at least one processor generates the determination data by using an amplitude value of measurement data for the determination target, the measurement data being measured by a gas sensor that is among the plurality of gas sensors and that has lower specificity than a predetermined criterion to a plurality of measurement targets which have been subjected to measurement by the plurality of gas sensors, to normalize the measurement data measured by the plurality of gas sensors.

3. The anomaly determination apparatus according to claim 1, wherein the at least one processor further carries out a normalization process,
the reference data is data that is obtained in accordance with a relationship between measurement data from the plurality of gas sensors for gas generated from a learning target which is normal, and
in the normalization process, the at least one processor generates the reference data by using an amplitude value of measurement data for the learning target, the measurement data being measured by a gas sensor that is among the plurality of gas sensors and that has lower specificity than a predetermined criterion to a plurality of measurement targets which have been subjected to measurement by the plurality of gas sensors, to normalize the measurement data measured by the plurality of gas sensors for the learning target.

4. The anomaly determination apparatus according to claim 1, wherein
in the determination process, the at least one processor uses a local outlier factor method to determine an anomaly regarding the determination target.

5. The anomaly determination apparatus according to claim 4, wherein
the reference data is data that is obtained in accordance with a relationship between measurement data from the plurality of gas sensors for gas generated from a learning target which is normal,
the at least one processor further carries out a threshold determination process for determining a threshold from a distribution of the reference data,
in the determination process, the at least one processor determines the anomaly regarding the determination target with use of the local outlier factor method in which the threshold is used.

6. An anomaly detection apparatus comprising:
a heating and burning section that heats and burns a determination target so as to generate gas;
a plurality of gas sensors that carry out measurement with respect to the gas and that have different responsivities from each other depending on a composition of gas;
a display section; and
an anomaly determination apparatus according to claim 1, wherein, in a case where it is determined that there is an anomaly in the determination target, the at least one processor further carries out a display control process for causing the display section to display an indication indicating that there is an anomaly in the determination target.

7. An anomaly determination method comprising:
acquiring, from a plurality of gas sensors having different responsivities from each other depending on a composition of gas, measurement data for gas generated from a determination target; and
determining an anomaly regarding the determination target by comparing reference data with determination data that is obtained from the measurement data from the respective plurality of gas sensors for the determination target and that is obtained in accordance with a relationship between the measurement data.

8. A non-transitory computer-readable storage medium storing therein A an anomaly determination program for causing a computer to carry out:
an acquisition process for acquiring, from a plurality of gas sensors having different responsivities from each other depending on a composition of gas, measurement data for gas generated from a determination target; and
a determination process for determining an anomaly regarding the determination target by comparing reference data with determination data that is obtained from the measurement data from the respective plurality of gas sensors for the determination target and that is obtained in accordance with a relationship between the measurement data.
